(19) United States
(12) Patent Application Publication
(10) Pub. No.: US 2022/0387820 A1
(43) Pub. Date: Dec. 8, 2022

(54) SAFETY MONITORING AND MANAGEMENT SYSTEM FOR PHOTOTHERAPY MACHINE

(71) Applicants: CHEN YA LIU, TAOYUAN CITY (TW); CHIN YUAN LIU, TAOYUAN CITY (TW)

(72) Inventors: CHEN YA LIU, TAOYUAN CITY (TW); CHIN YUAN LIU, TAOYUAN CITY (TW)

(21) Appl. No.: 17/828,519

(22) Filed: May 31, 2022

(30) Foreign Application Priority Data
Jun. 2, 2021 (TW) 110120075

(51) Int. Cl. A61N 5/06

(52) U.S. Cl. CPC A61N 5/0625; A61N 2005/0637; A61N 2005/0659; A61N 2005/0626

(57) ABSTRACT

A safety monitoring and management system for phototherapy machine, comprising: a cabin, the inside space is enough to containing a user body, at least one infrared light source arranged inside the cabin, using the thermal radiation energy generated by the infrared light source to irradiate the human body inside the cabin; a peripheral console is set around the phototherapy machine; a monitoring and management platform arranged at a position far from the phototherapy machine and the peripheral console; an online data integration system receives the information uploaded by the peripheral console or the monitoring and management platform through the Internet, for the user to adjust the parameter of the infrared light therapy by himself.

### BACKGROUND OF THE INVENTION

### 1. Field of the Invention

The invention relates to a safety monitoring and management system for phototherapy machine, especially the one that has a safety monitoring and management system with peripheral and remote simultaneously monitoring the physiological condition of a user's body and the irradiation temperature.

### 2. Description of the Related Art

Infrared rays penetrate into the depths of 40 mm of human skin. By promoting the resonance of human cells, the electrification and movement expand blood vessels, so as to smooth blood circulation and generate self-heating, increase the temperature deep inside the body, and enhance metabolism, activate the cell tissue, promote the excretion of toxins and wastes in the body and relieve fatigue. When the temperature of the body part increases, it can inhibit the proliferation of cancer cells and activate the immune system. Moreover, when the body temperature increases by 1° C., the immune system is activated 5 to 6 times. Therefore, by increasing the body temperature, it can relieve pain and prevent the proliferation of cancer cells, and activate the immune system to reduce cancer cells.

I own a Taiwan Patent, No. 109215832 disclosed an infrared device with color temperature monitoring function, it uses infrared thermal radiation energy to irradiate the human body, and let the thermal effect of radiant energy to warm the human skin surface and superficial muscles and blood vessels, which can achieve the purpose of improving local blood circulation and relieving muscle fatigue and soreness. A phototherapy machine that uses infrared thermal radiation energy to irradiate the human body is let the whole body of the human body be placed in the cabin, and the human body is irradiated with thermal radiation energy with a temperature of 40° C. 45° C., so as to get the best effect; however, the user will feel bored when lying in the cabin for a long time; more importantly, when the phototherapy machine irradiates the human body with infrared rays, there is no monitoring device to detect the physiological condition of the user, so it cannot know the physiological condition of the user's body in time, such as: heartbeat value, blood oxygen value, etc.; therefore, there is a risk of safety in use.

When it is detected that the physiological condition of the user's body is abnormal, or the temperature in the phototherapy machine cabin is too high, if there is no staff next to the phototherapy machine, the cabin door and the infrared irradiation device of the phototherapy machine cannot be opened immediately; And there is a risk of safety in use, and there is still room for improvement.

### SUMMARY OF THE INVENTION

It is a primary objective of the present invention to provide a safety monitoring and management system for phototherapy machine that having the function of monitoring the physiological condition and irradiation temperature of the user's body at the peripheral and remote ends at the same time, and when the user's heartbeat value, blood oxygen value, etc. are found to be abnormal or the infrared irradiation temperature is abnormal, the cabin door will be automatically opened immediately and turn off all infrared irradiation devices of the phototherapy machine to ensure the safety of users.

It is another objective of the present invention is to provide a safety monitoring and management system for phototherapy machine that can receive the information uploaded by the peripheral console or the monitoring and management platform through the Internet, as the parameter value for the user to adjust the infrared phototherapy course by himself, so as to improve the use effect of the phototherapy.

It is another objective of the present invention is to provide a safety monitoring and management system for phototherapy machine, wherein there is a media player in the cabin, so the user will not feel bored if he lies in the cabin for a long time, and the display screen of the media player can also display the user's own real-time physiological state, and the user can adjust the infrared light therapy at any time.

It is another objective of the present invention is to provide a safety monitoring and management system for phototherapy machine that having a fluid port for letting the fluid in the fluid port can perform temperature control and heat dissipation on the halogen bulb, thereby improving the efficiency of product reliability.

In order to achieve the above objectives, the present invention comprise: a phototherapy machine includes a cabin, the cabin having a cabin door and a cabin base plate, the inside space is enough to containing a user body, at least one infrared light source arranged inside the cabin, using the thermal radiation energy generated by the infrared light to irradiate the human body inside the cabin; wherein the cabin further includes a physiological monitoring device configured on the user for monitoring the physiological characteristics of the user to generate a physiological signal; the upper end of the cabin door the is pivoted on the two shaft seat on the left and right sides of the cabin by a pivot shaft, and the lower end is in a state that can be opened upward and closed downward; a first signal transmission unit, which is electrically connected to the physiological monitoring device for sending the physiological signal of the user; a media player, arranged in the cabin and facing the user, the media player has a second signal transmission unit for receiving the user's physiological signal sent by the first signal transmission unit; a first processing module, which is electrically connected to the second signal transmission unit; a first display unit electrically connected to the first processing module for providing entertainment images, the first processing module displays the physiological signal of the user digitally or graphically on the first display unit, and when it is determined that the physiological characteristics of the user are abnormal, a first warning unit outputs a warning signal; a third signal transmission unit is electrically connected to the first processing module for sending out the physiological signal and warning signal of the user; a motor driving unit is electrically connected to the third signal transmission unit, which is arranged on one side of the left and right sides of the cabin and adjacent to the cabin door for driving the cabin door to be opened upward and closed downward, the driving unit consists of a deceleration motor, a driver and a sector gear, the output shaft of the deceleration motor is connected to a transmission gear, the top of the sector gear is connected to one end of the pivot shaft of the cabin door, and the bottom of the sector gear is a circular arc gears rack, and the circular arc gears rack is meshed with the transmission gear, and the deceleration motor drive the displacement of the sector gear, thereby driving the pivot shaft of the cabin door to rotate; a peripheral console is set around the phototherapy machine, the peripheral console has a fourth signal transmission unit for receiving the signal sent by the third signal transmission unit; a second processing module is electrically connected the fourth signal transmission unit, a safety control unit is electrically connected to the second processing module, having a safety value parameter preset in the second processing module, when the fourth signal transmission unit receives the physiological warning signal of the user transmitting by the third signal transmission unit, controls the motor driving unit to start to open the cabin door and close all the infrared light source irradiation devices of the phototherapy machine; a second display unit is electrically connected to the second processing module for displaying the physiological signal of the user in the cabin and the operating state of the phototherapy machine; a second warning unit is electrically connected to the second processing module for displaying the physiological warning signal of the user; a first long range wireless transmission unit is electrically connected to the second processing module; a monitoring and management platform arranged at a position far from the phototherapy machine and the peripheral console, having a second long range wireless transmission unit for receiving signals sent by the first long range wireless transmission unit; a central processing unit is electrically connected to the second long range wireless transmission unit, a screen is electrically connected to the central processing unit; a database is electrically connected to the central processing unit for storing the user's physiological information; and when the monitoring and management platform finds that the physiological state of the user is abnormal or the temperature of the cabin is too high, it immediately controls the motor driving unit through the second processing module and the safety control unit of the peripheral console to actuate and drive the cabin door to open and close all infrared light source irradiation devices of the phototherapy machine; and an online data integration system receives the information uploaded by the peripheral console or the monitoring and management platform through the Internet, collects and analyzes the physiological state of the user through big data, and obtain the integrated analysis data from the peripheral console or the monitoring and management platform of the online data integration system, for the user to adjust the parameter of the infrared light therapy by himself.

Also, in a preferred embodiment, the monitoring and management platform is set to monitor multiple phototherapy machines and peripheral consoles at the same time.

Also, in a preferred embodiment, further includes a temperature detection unit, which is electrically connected to the first processing module for providing the infrared irradiation temperature inside the cabin, and the infrared irradiation temperature is displayed simultaneously through the first display unit of the cabin, the second display unit of the peripheral console and the screen of the monitoring and management platform, and the safety control unit monitors whether the temperature is abnormal.

Also, in a preferred embodiment, the physiological monitoring device is a wearable physiological signal sensing component, including: a smart watch for detecting pulse and blood oxygen, a heartbeat detection belt tied to the chest, a blood pressure measuring device set on the arm, a finger clamp pulse oximeter, or any other combination thereof.

Also, in a preferred embodiment, the first, second, third and fourth signal transmission units include wired transmission and wireless transmission.

Also, in a preferred embodiment, the first warning unit is arranged on the cabin, including an alarm, a flashing light, a display unit sound or a prompt window, or any other combination thereof.

Also, in a preferred embodiment, the first display unit and the second display unit include a touch interface or an operation interface.

Also, in a preferred embodiment, the first processing module and the second processing module include a microprocessor and a memory.

Also, in a preferred embodiment, the number of the infrared light source is plural, and the infrared light sources are respectively installed on both sides of the interior of the cabin and below the bottom of the cabin base plate or above the interior of the cabin to form a multi-faceted illumination light source.

Also, in a preferred embodiment, the bottom of the cabin base plate is able to be set inclined and set with a sweat collection port at the lower end, and a sweat collection box is arranged below the bottom of the cabin base plate relative to the sweat collection port.

Also, in a preferred embodiment, the infrared light sources include a flow heat exchanger, a first end pipeline joint of the flow heat exchanger is combined with the first fluid port of the infrared light source, so that the filtrate enter the containing space through the first fluid port, and a second end pipeline joint of the flow heat exchanger is combined with the second fluid port, so that the filtrate flow out of the containing space and return to the flow heat exchanger to form a circulating temperature control and cooling system.

With the feature disclosed above, the present invention utilizes the thermal radiation energy generated by the infrared light source to irradiate the human body inside the cabin; and uses the peripheral console and the monitoring and management platform at the remote position to monitor the physiological condition and irradiation temperature of the user's body at the peripheral and remote ends at the same time, and when the user's heartbeat value, blood oxygen value, etc. are found to be abnormal or the infrared irradiation temperature is abnormal, the cabin door will be automatically opened immediately and turn off all infrared irradiation devices of the phototherapy machine to ensure the safety of users. And through the online data integration system, the information uploaded by the peripheral console or the monitoring and management platform is received through the Internet as a parameter value for the user to adjust the infrared phototherapy course, thereby improving the use effect of the phototherapy.

### BRIEF DESCRIPTION OF THE DRAWINGS

FIG. 1 is a schematic diagram illustrating the safety monitoring and management system of the preferred embodiment of the present invention;

FIG. 2 is a block diagram illustrating the main structure of the preferred embodiment of the present invention;

FIG. 3 is a perspective views of the main structure of the preferred embodiment of the present invention;

FIG. 4 is a perspective views of the structure of the phototherapy machine of the present invention while the cabin door is closed;

FIG. 5 is a perspective views of the structure of the phototherapy machine of the present invention while the cabin door is opened;

FIG. 6A is a partially sectional view of the phototherapy machine of the present invention;

FIG. 6B is a partially sectional view of the phototherapy machine of the present invention;

FIG. 6C is a perspective views of the motor driving unit of the present invention;

FIG. 7 is a sectional view of the phototherapy machine of the present invention;

FIG. 8A is a schematic diagram illustrating the application of the phototherapy machine of the present invention while the cabin door is closed;

FIG. 8B is a schematic diagram illustrating the application of the phototherapy machine of the present invention while the cabin door is opened;

FIG. 9 is a schematic diagram illustrating the structure of the infrared light source of the present invention;

FIG. 10 is a schematic diagram illustrating the structure of the infrared light source of the present invention;

FIG. 11 is a schematic diagram illustrating the combination of the lamp holders and the halogen bulbs of the infrared light source of the present invention;

FIG. 12 is a side view of the of the infrared light source of the present invention;

FIG. 13 is the sectional view along line 13-13 in FIG. 12;

FIG. 14 is a side view of the of the infrared light source of the present invention;

FIG. 15 is the sectional view along line 15-15 in FIG. 14.

### DETAILED DESCRIPTION OF THE PREFERRED EMBODIMENT

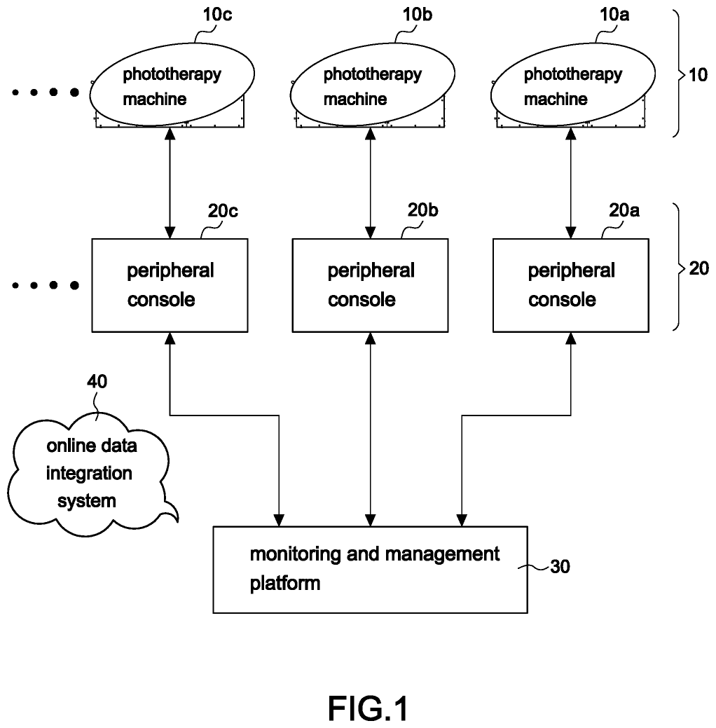

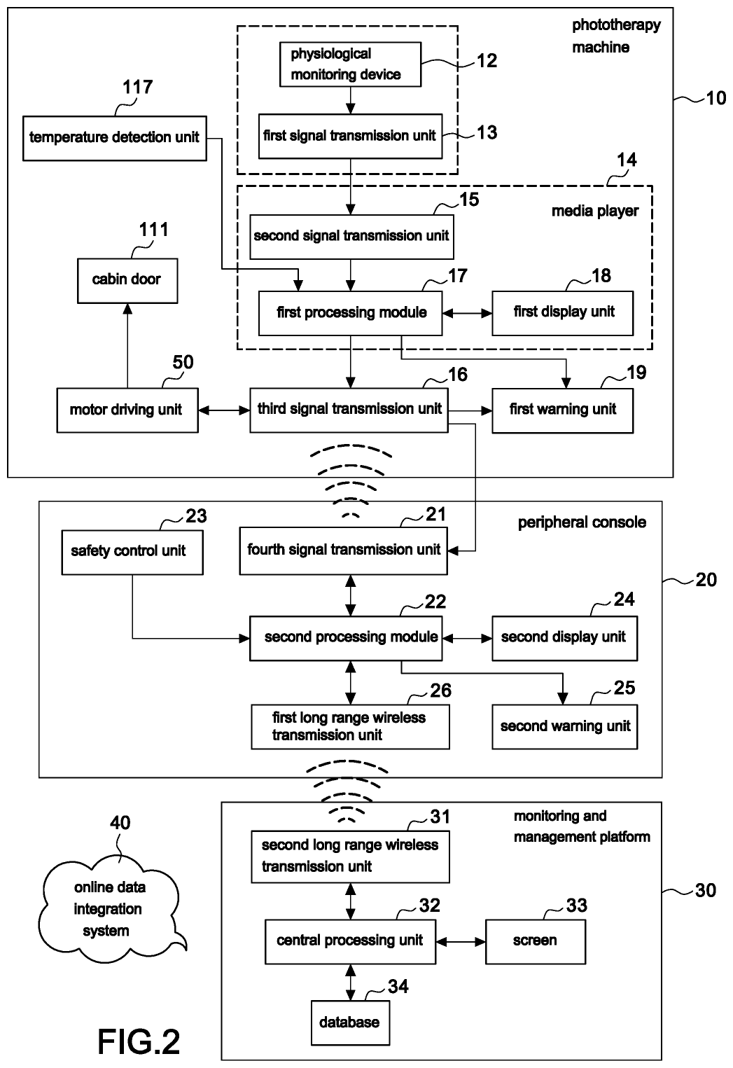

Referring to FIGS. 1-8, the present invention includes: a phototherapy machine 10 includes a cabin 11, the cabin 11 having a cabin door 111 and a cabin base plate 112, the inside space is enough to containing a user body, at least one infrared light source 60 arranged inside the cabin 11, using the thermal radiation energy generated by the infrared light source 60 to irradiate the human body inside the cabin 11.

The main feature of the present invention is: the cabin 11 further includes a physiological monitoring device 12 configured on the user for monitoring the physiological characteristics of the user to generate a physiological signal; in this embodiment, the physiological monitoring device 12 is a wearable physiological signal sensing component, including: a smart watch for detecting pulse and blood oxygen, a heartbeat detection belt tied to the chest, a blood pressure measuring device set on the arm, a finger clamp pulse oximeter, or any other combination thereof. Since the physiological monitoring device 12 is a mature product in the market, so its structure will not be described in detail. The present invention uses this wearable physiological signal sensing component to detect the user's pulse, heartbeat, blood pressure, heart rhythm and blood oxygen and other physiological signals, so as to obtain the user's physiological condition at any time, and when the user's physiological condition is found to be abnormal, the cabin door will be automatically opened and all infrared irradiation devices of the phototherapy machine will be automatically closed to ensure the safety of users.

A media player 14, arranged in the cabin 11 and facing the user, the media player 14 has a second signal transmission unit 15 for receiving the user's physiological signal sent by the first signal transmission unit 13; a first processing module 17, which is electrically connected to the second signal transmission unit 15; a first display unit 18 electrically connected to the first processing module 17 for providing entertainment images, the first processing module 17 displays the physiological signal of the user digitally or graphically on the first display unit 18, and when it is determined that the physiological characteristics of the user are abnormal, a first warning unit 19 outputs a warning signal; a third signal transmission unit 16 is electrically connected to the first processing module 17 for sending out the physiological signal and warning signal of the use.

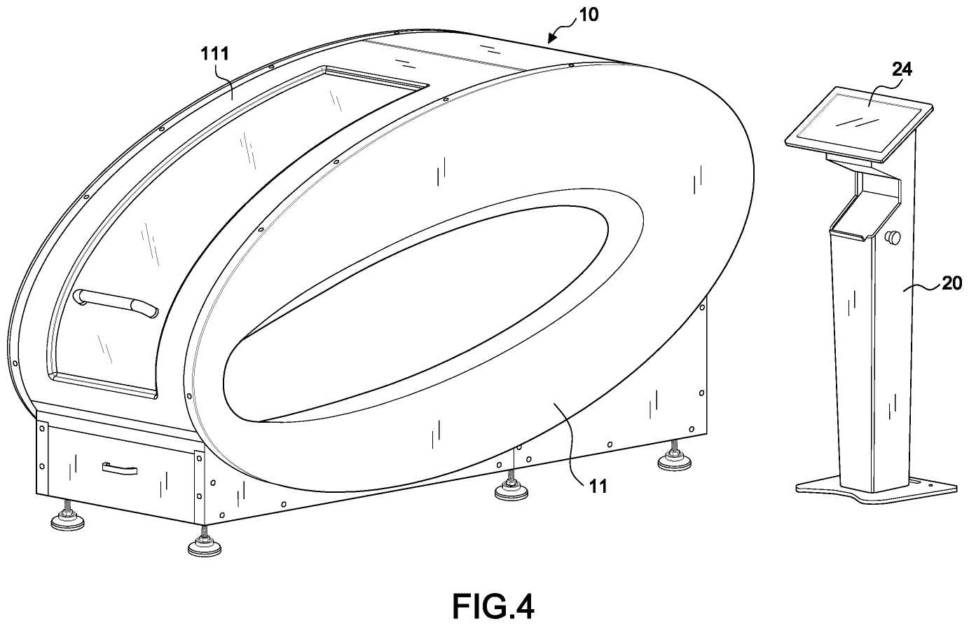

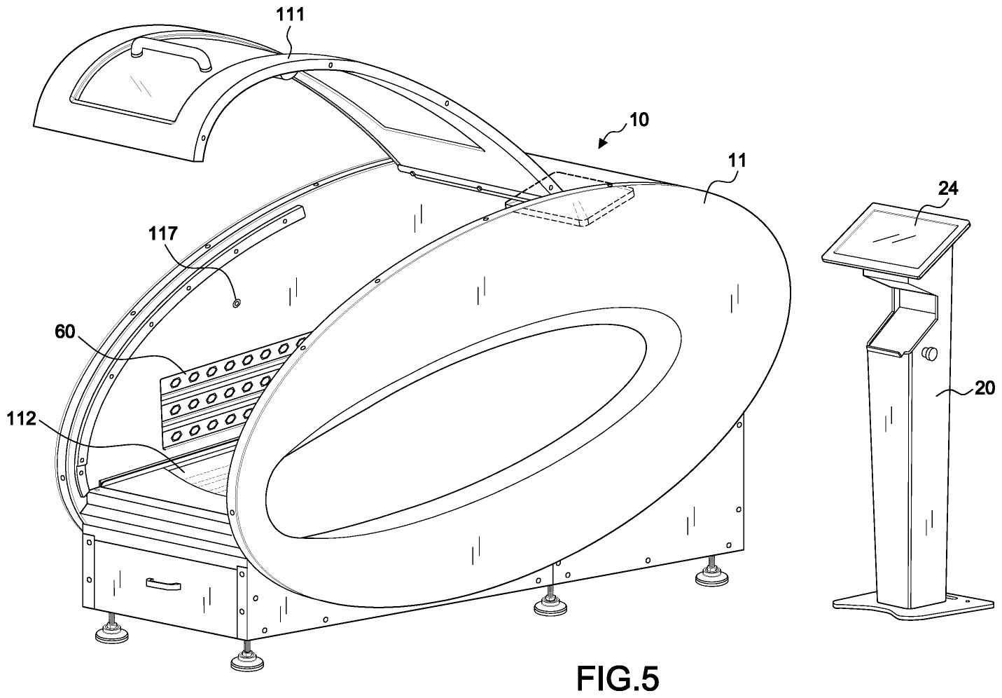

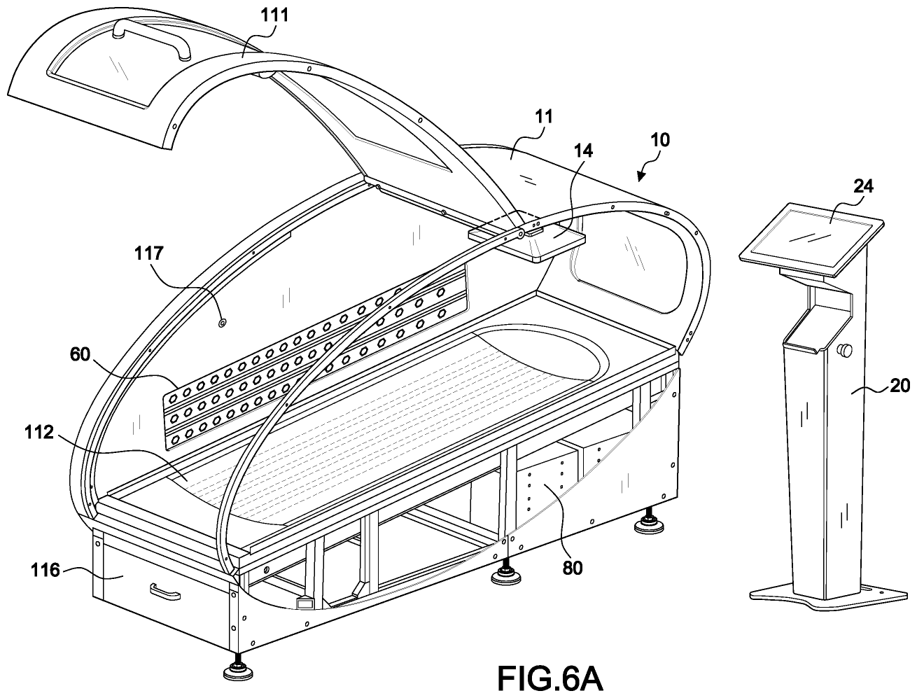

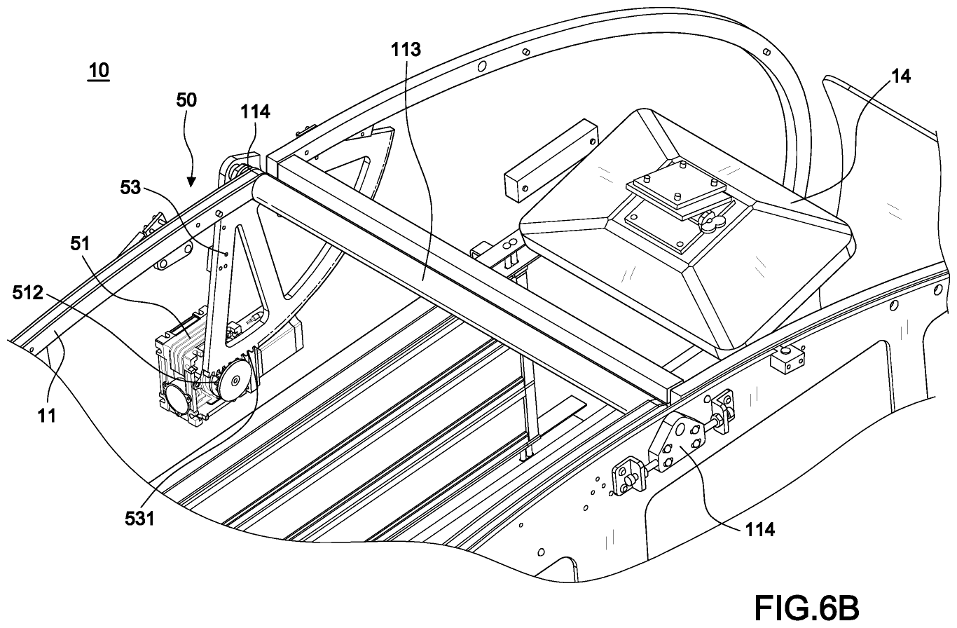

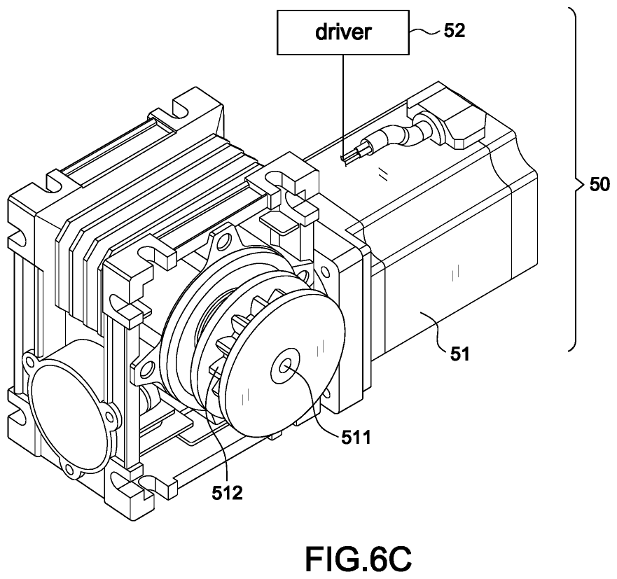

Referring to FIGS. 6B-6C, a motor driving unit 50 is electrically connected to the third signal transmission unit 16, which is arranged on one side of the left and right sides of the cabin 11 and adjacent to the cabin door 111 for driving the cabin door 111 to be opened upward and closed downward, the driving unit 50 consists of a deceleration motor 51, a driver 52 and a sector gear 53, the output shaft 511 of the deceleration motor 51 is connected to a transmission gear 512, the top of the sector gear 53 is connected to one end of the pivot shaft 113 of the cabin door 111, and the bottom of the sector gear 53 is a circular arc gears rack 531, and the circular arc gears rack 531 is meshed with the transmission gear 512, when the driver 52 receives the start signal, the deceleration motor 51 drive the displacement of the sector gear 53, thereby driving the pivot shaft 113 of the cabin door 111 to rotate; FIG. 4 is showing when the cabin door 111 is closed. FIG. 5 is showing when the cabin door 111 is opened.

Referring to FIGS. 1-3, a peripheral console 20 is set around the phototherapy machine 10, the peripheral console 20 has a fourth signal transmission unit 21 for receiving the signal sent by the third signal transmission unit 16; a second processing module 22 is electrically connected the fourth signal transmission unit 21, a safety control unit 23 is electrically connected to the second processing module 22, having a safety value parameter preset in the second processing module 22, when the fourth signal transmission unit 21 receives the physiological warning signal of the user transmitting by the third signal transmission unit 16, controls the motor driving unit 50 to start to open the cabin door 111; a second display unit is electrically connected to the second processing module 22 for displaying the physiological signal of the user in the cabin 11 and the operating state of the phototherapy machine 10; a second warning unit 25 is electrically connected to the second processing module 22 for displaying the physiological warning signal of the user; a first long range wireless transmission unit 26 is electrically connected to the second processing module 22; in this embodiment, the first, second, third and fourth signal transmission units include wired transmission and wireless transmission, and the first processing module 17 and the second processing module 21 include a microprocessor and a memory.

A monitoring and management platform 30 arranged at a position far from the phototherapy machine 10 and the peripheral console 20, having a second long range wireless transmission unit 31 for receiving signals sent by the first long range wireless transmission unit 26; a central processing unit 32 is electrically connected to the second long range wireless transmission unit 31, a screen 33 is electrically connected to the central processing unit 32; a database 34 is electrically connected to the central processing unit 32 for storing the user's physiological information; and when the monitoring and management platform 30 finds that the physiological state of the user is abnormal or the temperature of the cabin 11 is too high, it immediately controls the motor driving unit 50 through the second processing module 22 and the safety control unit 23 of the peripheral console 20 to actuate and drive the cabin door 111 to open and close all infrared light source irradiation devices of the phototherapy machine 10.

An online data integration system 40 receives the information uploaded by the peripheral console 20 or the monitoring and management platform 30 through the Internet, collects and analyzes the physiological state of the user through big data, and obtain the integrated analysis data of the online data integration system 40 through the peripheral console 20 or the monitoring and management platform 30, for the user to adjust the parameter of the infrared light therapy by himself.

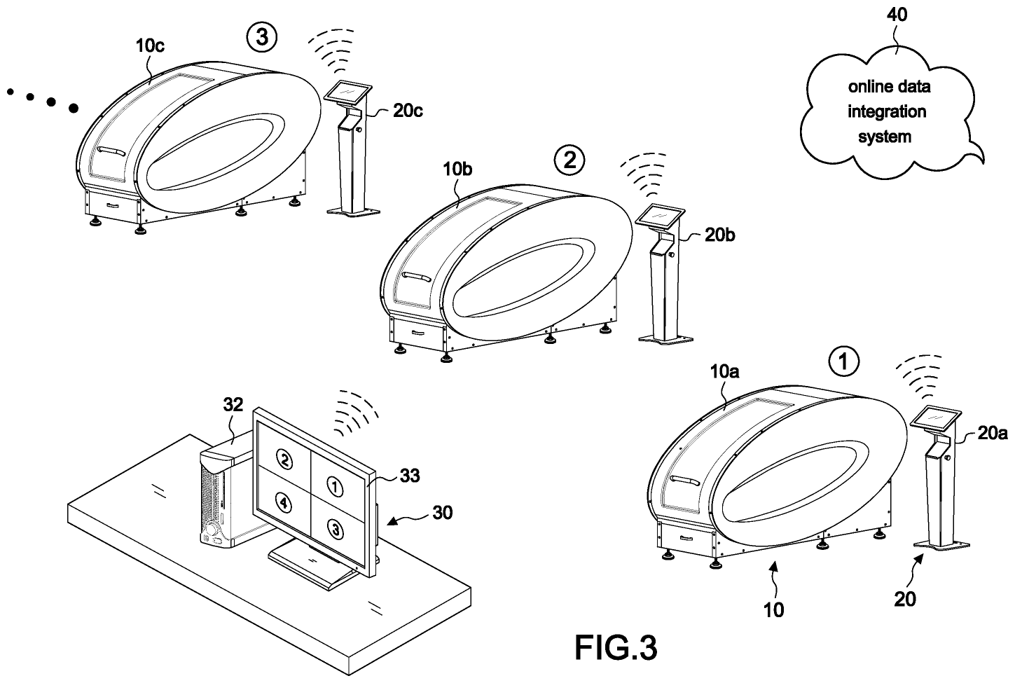

In this embodiment, the monitoring and management platform 30 is set to monitor a plurality of phototherapy machines 10 and peripheral consoles 20 at the same time. As FIG. 3 showing, there are several phototherapy machines 10 (10a, 10b, 10c . . . ) and several peripheral consoles 20 (20a, 20b, 20c . . . ), but only one monitoring and management platform 30 is needed to monitor them all.

In this embodiment, further includes a temperature detection unit 117, which is electrically connected to the first processing module 17 for providing the infrared irradiation temperature inside the cabin 11, and the infrared irradiation temperature is displayed simultaneously through the first display unit 18 of the cabin 11, the second display unit 24 of the peripheral console 20 and the screen 33 of the monitoring and management platform 30, and the safety control unit 23 monitors whether the temperature is abnormal. In this embodiment, the first display unit 18 and the second display unit 24 include a touch interface or an operation interface.

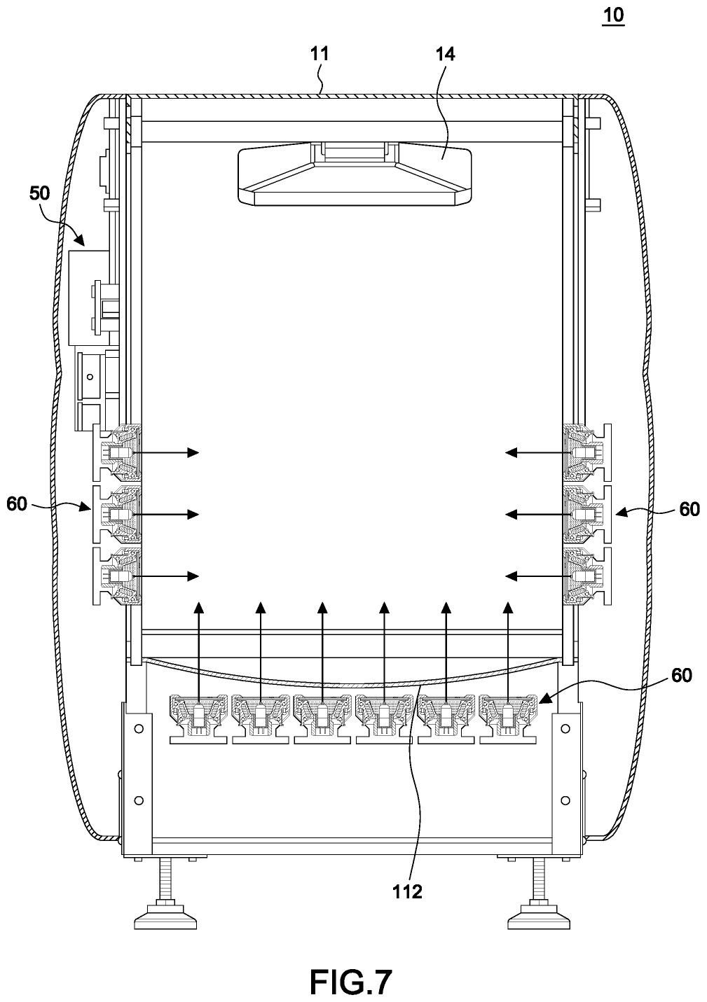

Referring to FIG. 7, in this embodiment, the number of the infrared light source 60 is plural, and the infrared light sources are respectively installed on both sides of the interior of the cabin 11 and below the bottom of the cabin base plate 112 or above the interior of the cabin 11 to form a multi-faceted illumination light source.

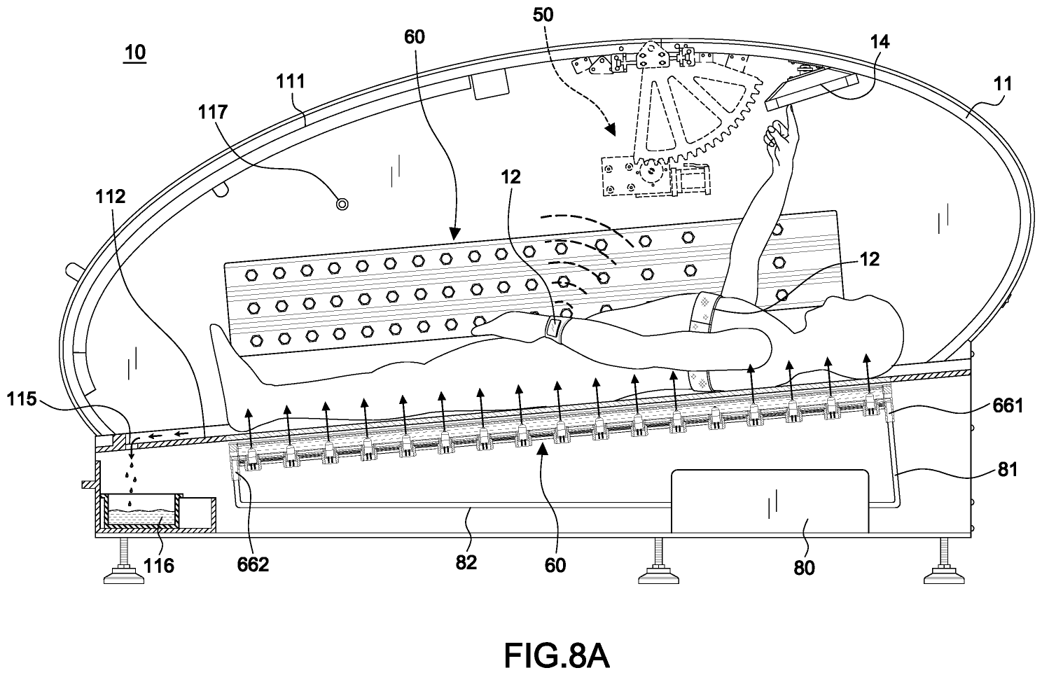

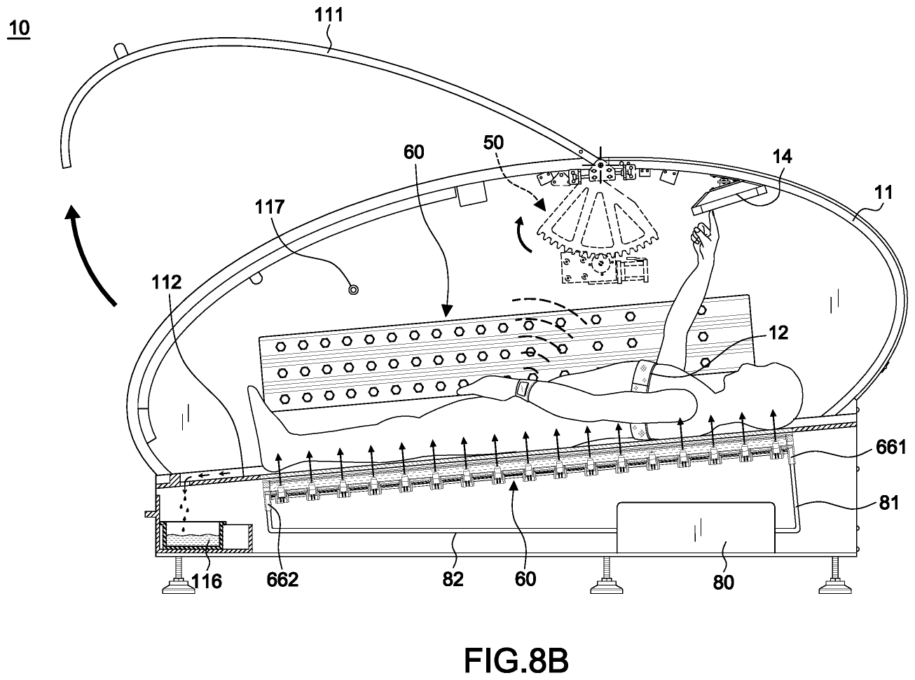

Referring to FIG. 8A and FIG. 8B, in this embodiment, the bottom of the cabin base plate 112 is able to be inclined and set with a sweat collection port 115 at the lower end, and a sweat collection box 116 is arranged below the bottom of the cabin base plate 112 relative to the sweat collection port 115. With the feature disclosed above, the present invention install a plurality of infrared light sources 60 are inside the cabin 11, and the whole lying body of the user can be accommodated inside the cabin 11, so the thermal radiation energy generated by the infrared light sources 60 can be used to irradiate the human body inside the cabin 11, and having the effect of irradiating infrared rays to the whole body at the same time; furthermore, the sweat discharged by the human body due to the irradiation of infrared rays flows to the sweat collection port 115 at the lower end of the cabin base plate 112 by gravity, and drips into the sweat collection box 116 to be collected, having the effect of collecting the sweat discharged by the human body due to the irradiation of infrared rays; moreover, the sweat collected by the present invention is discharged by irradiating infrared rays, and the factors that cause perspiration (the amount of exercise does not change and the temperature does not change) do not change, as a result, the information representing the health status of the body through the large amount of sweat collected is extremely stable, and the sufficient amount of sweat and the factors of perspiration remain unchanged, which helps to improve the accuracy of the sweat test.

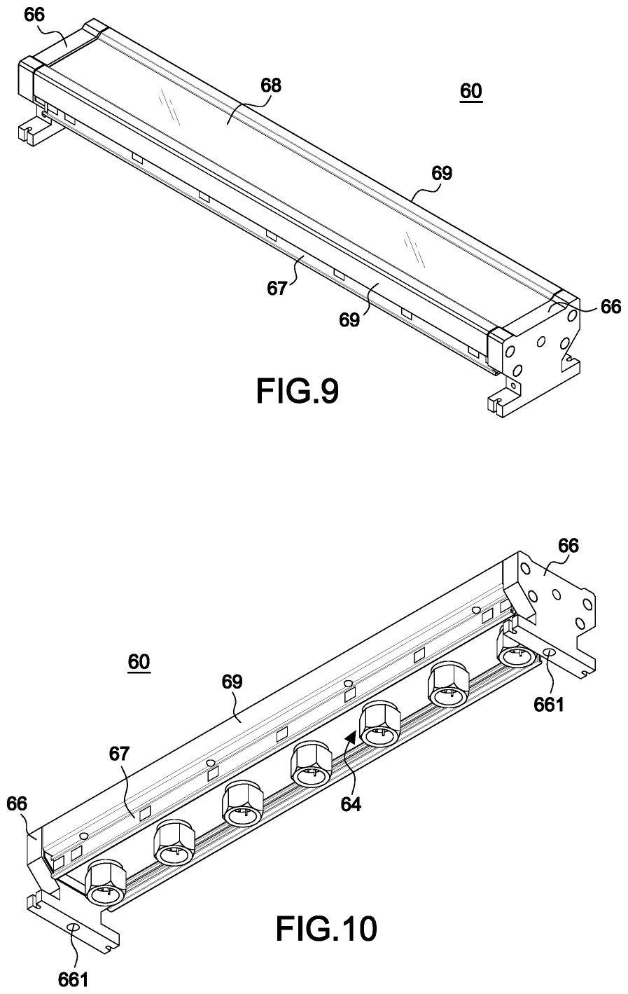

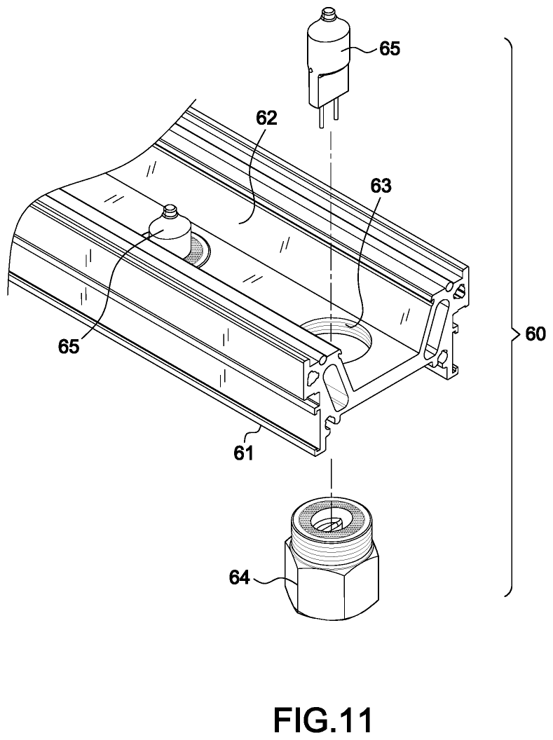

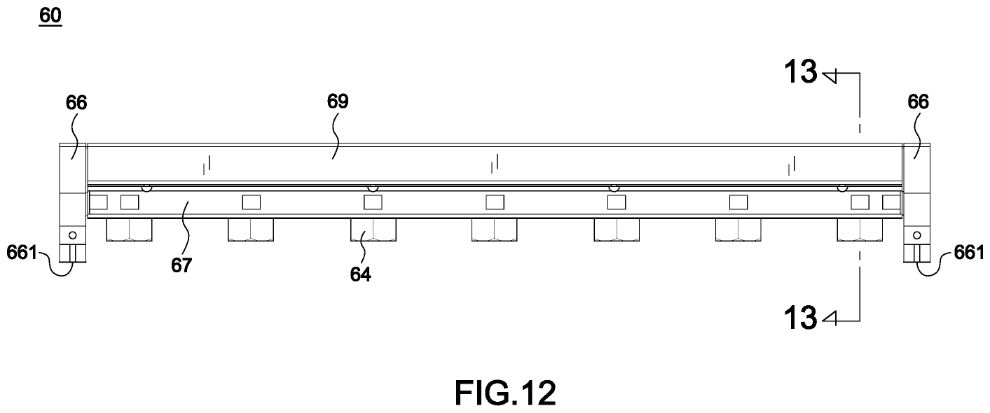

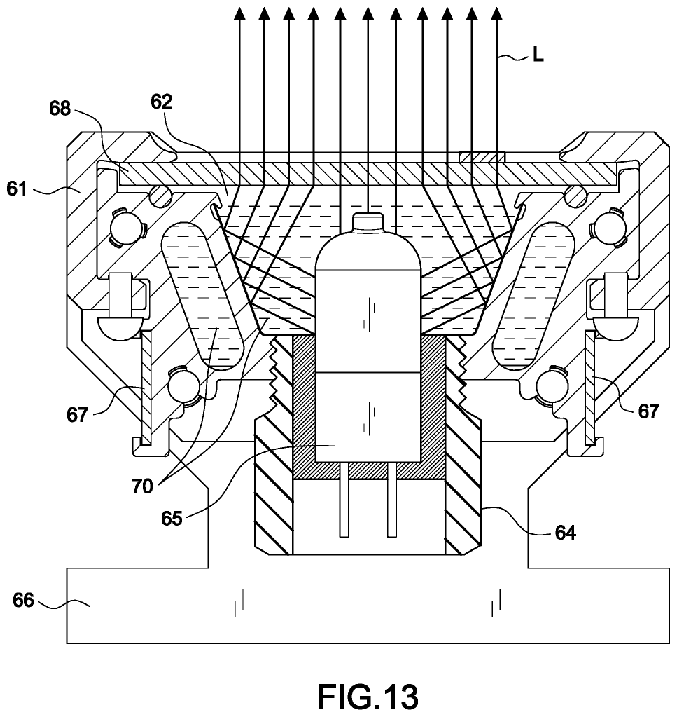

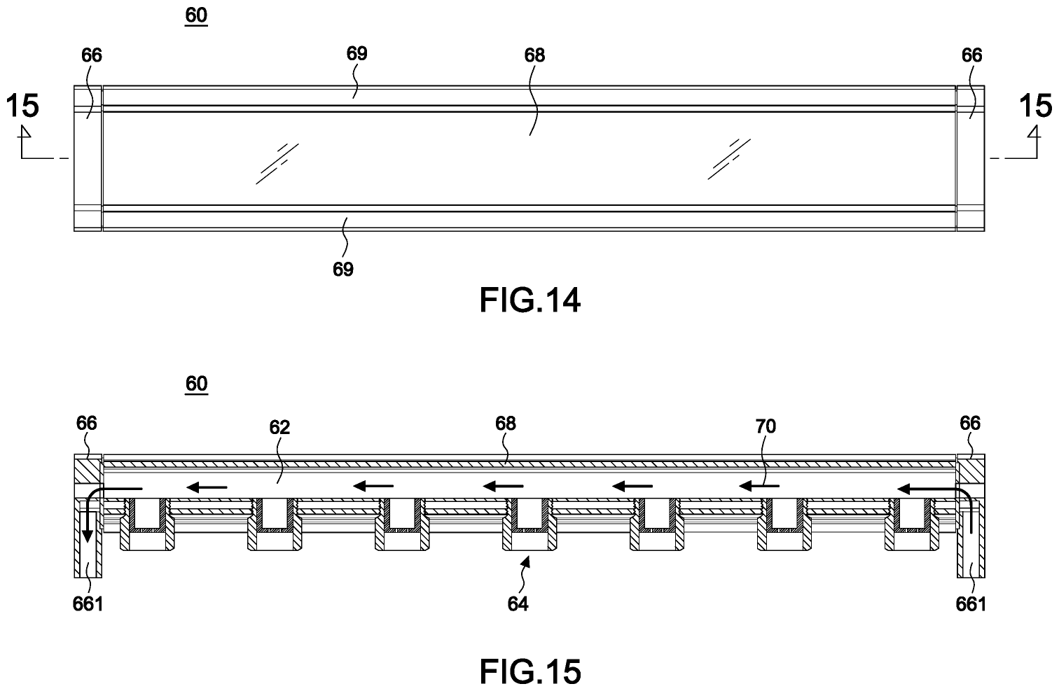

Referring to FIGS. 9-15, in this embodiment, a main body 61, a long body with a concave section is extruded from aluminum, having a containing space 62 forming inside, and at least a through hole 63 at the bottom of the main body 61; at least a lamp holder 64, combined to the through hole 63 that arranged at the bottom of the main body 61; at least a halogen bulb 65, combined to the lamp holder 64, and the halogen bulb 65 arranged inside the containing space 62; two supports 66 are respectively combined with the two end faces of the main body 61, and the two supports 66 are respectively set with a first fluid port 661 and a second fluid port 662 which are in communication with the containing space 62; a light guide board 68 combined on the top of the main body 61; two joints strips 69 are respectively combined with the two long sides of the main body 61, so that the light guide board 68 is fixed on the top of the main body 61; a filtrate 70 is filled in the containing space 62, let the halogen bulb 65 arrange in the filtrate 70, for making the full-wavelength color light emitting by halogen bulb 65 change the color temperature after passing through the filtrate 70 to form a mixed light that is irradiated outside the light guide board 68; at least a printed circuit board assembly 67 electrically connected to the lamp holder 64 for switching the halogen bulb 65, and the printed circuit board assembly 67 is used to control the color temperature of the mixed light to maintain the set infrared color temperature. As for the technical means of color temperature monitoring, I have disclosed in the previous Taiwan patent No. 109215832 “an infrared device with color temperature monitoring function”, since it is not the subject of the patent of the present invention, it will not be described in detail.

Moreover, as FIG. 8A showing, in this embodiment, further includes a flow heat exchanger 80, a first end pipeline joint of the flow heat exchanger 80 is combined with the first fluid port 661 of the infrared light source, so that the filtrate 70 enter the containing space 62 through the first fluid port 661, and a second end pipeline joint 82 of the flow heat exchanger 80 is combined with the second fluid port 662, so that the filtrate 70 flow out of the containing space 62 and return to the flow heat exchanger 80 to form a circulating temperature control and cooling system. Therefore, the filtrate 70 not only makes making the full-wavelength color light emitting by halogen bulb 65 change the color temperature after passing through the filtrate 70 to form a mixed light that is irradiated outside the light guide board 68, but also forms a circulating temperature control and heat dissipation system by the continuous circulating flow of the filtrate 70 and return to the flow heat exchanger 80, which can ensure that the temperature of the halogen bulb 65 will not be overheated, and has the effect of improving product reliability and service life.

With the features disclosed above, the present invention utilizes the thermal radiation energy generated by the infrared light source 60 to irradiate the human body inside the cabin 11; and uses the peripheral console 20 and the monitoring and management platform 30 at the remote position to monitor the physiological condition and irradiation temperature of the user's body at the peripheral and remote ends at the same time, and when the user's heartbeat value, blood oxygen value, etc. are found to be abnormal or the infrared irradiation temperature is abnormal, the cabin door 11 will be automatically opened immediately and turn off all infrared irradiation devices of the phototherapy machine 10 to ensure the safety of users. And through the online data integration system 40, the information uploaded by the peripheral console 20 or the monitoring and management platform 30 is received through the Internet as a parameter value for the user to adjust the infrared phototherapy course, thereby improving the use effect of the phototherapy.

Although particular embodiments of the invention have been described in detail for purposes of illustration, various modifications and enhancements may be made without departing from the spirit and scope of the invention.

Accordingly, the invention is not to be limited except as by the appended claims.

What is claimed is:

1. A safety monitoring and management system for phototherapy machine, comprising:
a phototherapy machine includes a cabin, the cabin having a cabin door and a cabin base plate, the inside space is enough to containing a user body, at least one infrared light source arranged inside the cabin, using the thermal radiation energy generated by the infrared light source to irradiate the human body inside the cabin;
wherein the cabin further includes a physiological monitoring device configured on the user for monitoring the physiological characteristics of the user to generate a physiological signal;
the upper end of the cabin door the is pivoted on the two shaft seat on the left and right sides of the cabin by a pivot shaft, and the lower end is in a state that can be opened upward and closed downward;
a first signal transmission unit, which is electrically connected to the physiological monitoring device for sending the physiological signal of the user;
a media player, arranged in the cabin and facing the user, the media player has a second signal transmission unit for receiving the user's physiological signal sent by the first signal transmission unit; a first processing module, which is electrically connected to the second signal transmission unit; a first display unit electrically connected to the first processing module for providing entertainment images, the first processing module displays the physiological signal of the user digitally or graphically on the first display unit, and when it is determined that the physiological characteristics of the user are abnormal, a first warning unit outputs a warning signal; a third signal transmission unit is electrically connected to the first processing module for sending out the physiological signal and warning signal of the user; the first warning unit is arranged on the cabin;
a motor driving unit is electrically connected to the third signal transmission unit, which is arranged on one side of the left and right sides of the cabin and adjacent to the cabin door for driving the cabin door to be opened upward and closed downward, the driving unit consists of a deceleration motor, a driver and a sector gear, the output shaft of the deceleration motor is connected to a transmission gear, the top of the sector gear is connected to one end of the pivot shaft of the cabin door, and the bottom of the sector gear is a circular arc gears rack, and the circular arc gears rack is meshed with the transmission gear, and the deceleration motor drive the displacement of the sector gear, thereby driving the pivot shaft of the cabin door to rotate;
a peripheral console is set around the phototherapy machine, the peripheral console has a fourth signal transmission unit for receiving the signal sent by the third signal transmission unit; a second processing module is electrically connected the fourth signal transmission unit, a safety control unit is electrically connected to the second processing module, having a safety value parameter preset in the second processing module, when the fourth signal transmission unit receives the physiological warning signal of the user transmitting by the third signal transmission unit, controls the motor driving unit to start to open the cabin door and close all the infrared light source irradiation devices of the phototherapy machine; a second display unit is electrically connected to the second processing module for displaying the physiological signal of the user in the cabin and the operating state of the phototherapy machine; a second warning unit is electrically connected to the second processing module for displaying the physiological warning signal of the user; a first long range wireless transmission unit is electrically connected to the second processing module;
a monitoring and management platform arranged at a position far from the phototherapy machine and the peripheral console, having a second long range wireless transmission unit for receiving signals sent by the first long range wireless transmission unit; a central processing unit is electrically connected to the second long range wireless transmission unit, a screen is electrically connected to the central processing unit; a database is electrically connected to the central processing unit for storing the user's physiological information; and when the monitoring and management platform finds that the physiological state of the user is abnormal or the temperature of the cabin is too high, it immediately controls the motor driving unit through the second processing module and the safety control unit of the peripheral console to actuate and drive the cabin door to open and close all infrared light source irradiation devices of the phototherapy machine; and
an online data integration system receives the information uploaded by the peripheral console or the monitoring and management platform through the Internet, collects and analyzes the physiological state of the user through big data, and obtain the integrated analysis data of the online data integration system through the peripheral console or the monitoring and management platform, for the user to adjust the parameter of the infrared light therapy by himself.

2. The safety monitoring and management system for phototherapy machine as claimed in claim 1, wherein the monitoring and management platform is set to monitor multiple phototherapy machines and peripheral consoles at the same time.

3. The safety monitoring and management system for phototherapy machine as claimed in claim 1, wherein further includes a temperature detection unit, which is electrically connected to the first processing module for providing the infrared irradiation temperature inside the cabin, and the infrared irradiation temperature is displayed simultaneously through the first display unit of the cabin, the second display unit of the peripheral console and the screen of the monitoring and management platform, and the safety control unit monitors whether the temperature is abnormal.

4. The safety monitoring and management system for phototherapy machine as claimed in claim 1, wherein the physiological monitoring device is a wearable physiological signal sensing component, including: a smart watch for detecting pulse and blood oxygen, a heartbeat detection belt tied to the chest, a blood pressure measuring device set on the arm, a finger clamp pulse oximeter, or any other combination thereof.

5. The safety monitoring and management system for phototherapy machine as claimed in claim 1, wherein the first, second, third and fourth signal transmission units include wired transmission and wireless transmission.

6. The safety monitoring and management system for phototherapy machine as claimed in claim 1, wherein the first warning unit is arranged on the cabin, including an alarm, a flashing light, a display unit sound or a prompt window, or any other combination thereof.

7. The safety monitoring and management system for phototherapy machine as claimed in claim 1, wherein the first display unit and the second display unit include a touch interface or an operation interface.

8. The safety monitoring and management system for phototherapy machine as claimed in claim 1, wherein the first processing module and the second processing module include a microprocessor and a memory.

9. The safety monitoring and management system for phototherapy machine as claimed in claim 1, wherein the number of the infrared light source is plural, and the infrared light sources are respectively installed on both sides of the interior of the cabin and below the bottom of the cabin base plate or above the interior of the cabin to form a multi-faceted illumination light source.

10. The safety monitoring and management system for phototherapy machine as claimed in claim 1, wherein the bottom of the cabin base plate is able to be inclined and set with a sweat collection port at the lower end, and a sweat collection box is arranged below the bottom of the cabin base plate relative to the sweat collection port.

11. The safety monitoring and management system for phototherapy machine as claimed in claim 1, wherein the infrared light sources includes:
a main body, having a containing space forming inside, and at least a through hole;
at least a lamp holder, combined to the through hole that arranged at the bottom of the main body;
at least a halogen bulb, combined to the lamp holder, and the halogen bulb arranged inside the containing space;
two supports are respectively combined with the two end faces of the main body, and the two supports are respectively set with a first fluid port and a second fluid port which are in communication with the containing space;
a light guide board combined on the top of the main body;
two joints strips are respectively combined with the two long sides of the main body, so that the light guide board is fixed on the top of the main body;
a filtrate is filled in the containing space, let the halogen bulb arrange in the filtrate, for making the full-wavelength color light emitting by halogen bulb change the color temperature after passing through the filtrate to form a mixed light that is irradiated outside the light guide board;
at least a printed circuit board assembly electrically connected to the lamp holder for switching the halogen bulb; and;
a flow heat exchanger, a first end pipeline joint of the flow heat exchanger is combined with the first fluid port of the infrared light source, so that the filtrate enter the containing space through the first fluid port, and a second end pipeline joint of the flow heat exchanger is combined with the second fluid port, so that the filtrate flow out of the containing space and return to the flow heat exchanger to form a circulating temperature control and cooling system.
